(19) United States
(12) Patent Application Publication
(10) Pub. No.: US 2015/0324520 A1
(43) Pub. Date: Nov. 12, 2015

(54) Blood Glucose Meter Supporting Contextual Data Filtering

(71) Applicant: ROCHE DIAGNOSTICS OPERATIONS, INC., Indianapolis, IN (US)

(72) Inventors: Timothy N. Aykroyd, Carmel, IN (US); Amy C. Day, Fishers, IN (US); Paul J. Galley, Cumberland, IN (US); Erin K. McKinney, Brownsburg, IN (US); Mark G. Mears, Westfield, IN (US); Michelle M. Stevens, Fishers, IN (US); Nicole D. Tillery, Fishers, IN (US)

(73) Assignee: ROCHE DIAGNOSTICS OPERATIONS, INC., Indianapolis, IN (US)

(21) Appl. No.: 14/275,455

(22) Filed: May 12, 2014

(51) Int. Cl. G06F 19/28; G06F 17/30; G06F 19/00; A61B 5/145

(52) U.S. Cl. CPC G06F 19/28; A61B 5/14532; G06F 17/30353; G06F 17/30032; G06F 19/3406

(57) ABSTRACT

The handheld blood glucose meter creates blood glucose records having contextual information to improve filtering of blood glucose records for improved healthcare decisions. The contextual information includes events and associated sub-events. The user selects an event indicator from the plurality of event indicators, and the meter associates the blood glucose measurement with an event that corresponds to the event indicator. The user selects a sub-event indicator from the plurality of sub-event indicators, and the meter associates the blood glucose measurement with a sub-event that corresponds to the sub-event indicator. The user repeats the preceding steps to obtain a plurality of blood glucose measurements with associated events and sub-events. The user selects data filtering, a predetermined range of blood glucose measurements along with an event and sub-event to produce a filter result meeting these criteria.

### FIELD

This disclosure relates to handheld in vitro blood glucose meters that store and display blood glucose measurements and related data.

### BACKGROUND

Diabetes mellitus, often referred to as diabetes, is a chronic condition in which a person has elevated blood glucose levels that result from the body's inability to produce insulin, use insulin, or both. There are three main types of diabetes. Type 1 diabetes usually strikes children and young adults and is linked to conditions such as autoimmune, genetic, environmental, or a combination. Type 2 diabetes accounts for 90-95% of diabetes cases and is linked to obesity and physical inactivity. Gestational diabetes is a form of glucose intolerance diagnosed during pregnancy that usually resolves soon after delivery.

In 2013, some 382 million people worldwide are estimated to have diabetes, and an estimated 5.1 million people between the ages of 20 and 79 die from diabetes annually, according to the International Diabetes Foundation Diabetes Atlas. In the United States, nearly 24 million Americans have diabetes with an estimated 25 percent of seniors age 60 and older being affected, according to The Centers for Disease Control and Prevention. Diabetes costs are estimated to be $174 billion in the United States alone every year, according to the National Diabetes Information Clearinghouse. Without treatment, diabetes can lead to severe complications such as heart disease, stroke, blindness, kidney failure, amputations, and death related to pneumonia and flu.

Handheld blood glucose meters are used by persons with diabetes in a self-monitoring environment, such as at home and work, to periodically measure their blood glucose to obtain diagnostic information for therapy decisions. Blood glucose measurements are typically associated with a date and time but limited other information. For persons who test frequently, it is easy to get lost in the data because it can be difficult to select and view this data in a meaningful way. An example of a self-monitoring blood glucose meter is described in Roche, Accu-Chek® Nano Owner's Booklet for Self-Testing Only (2013). Other manufacturers of self-testing blood glucose meters include LifeScan, Inc. and Abbott Diabetes Care.

Some blood glucose meters provide pattern recognition that typically notify a user immediately when a current blood glucose measurement matches a pattern such as higher than the target range or lower than the target range and whether a collection of blood glucose measurement are trending toward higher than target range or trending toward lower than target range. Typically, blood glucose meter pattern recognition, once enabled, operates in the background on the meter without the need for user interaction other than to perhaps acknowledge when a pattern was recognized. An example of a handheld blood glucose meter with pattern recognition is described in the Life Scan, Inc., OneTouch Verio IQ Blood Glucose Monitoring System, Owner's Booklet (2011), and U.S. Patent Pub. No. 2013/0318439, Analyte Testing Method and System with High and Low Analyte Trends Notification, published Nov. 28, 2013, assigned to Life Scan, Inc.

There is a need for a handheld blood glucose meter that selectively filters blood glucose measurements for interpretation by the user for improved health care decisions.

### SUMMARY

A handheld blood glucose meter with contextual data filtering has a display and is operated by a user to obtain blood glucose measurements. The meter associates a date and time with each blood glucose measurement. The meter displays a plurality of event indicators on the meter display, and each of the event indicators are associated with a set of sub-event indicators, which are also known as contextual data. The user selects an event indicator from the plurality of event indicators, and the meter associates the blood glucose measurement with an event that corresponds to the event indicator. The meter displays a plurality of sub-event indicators associated with the selected event indicator. The user selects a sub-event indicator from the plurality of sub-event indicators, and the meter associates the blood glucose measurement with a sub-event that corresponds to the sub-event indicator. The user repeats the preceding steps to obtain a plurality of blood glucose measurements with associated events and sub-events.

To perform contextual filtering of the plurality of blood glucose measurements with associated events and sub-events, the user first selects data filtering on the meter display. Next, the user selects a predetermined range of blood glucose measurements, e.g. low blood glucose measurement or high blood glucose measurements. The user then selects an event indicator on the meter display and selects a sub-event indicator on the meter display that is associated with the event indicator. The meter displays a plurality of blood glucose measurements within the predetermined range that have both event indicator and sub-event indicator to aid the user in making healthcare decisions.

In other embodiments, the meter can have a plurality of blood glucose measurements stored that have an associated date and time along with an associated event and sub-event. The meter can then perform contextual filtering of blood glucose measurements without the need to obtain new blood glucose measurements as described above. In yet another embodiment, the meter can have a plurality of blood glucose measurements stored that have an associated date and time along with an associated event and sub-event. The meter transmits these blood glucose measurements to a computing device, such as a mobile phone, that performs contextual filtering of blood glucose measurements as described above.

### BRIEF DESCRIPTION OF THE DRAWINGS

FIG. 1 shows a self-monitoring environment for a person with diabetes;

FIG. 2 shows a blood glucose meter with a test strip and a computing device with a diabetes management application;

FIG. 3 shows an electrical block diagram of a handheld blood glucose meter;

FIG. 4 shows an electrical block diagram of a computing device;

FIG. 5 shows a flowchart for contextual data filtering on a handheld blood glucose meter;

FIG. 6 shows selected user interface navigation screens for selecting an event and a sub-event;

FIG. 7 shows a flowchart for contextual data filtering with a computing device; and

FIG. 8 shows selected user interface navigation screens for conditions that stop contextual data filtering.

### DETAILED DESCRIPTION

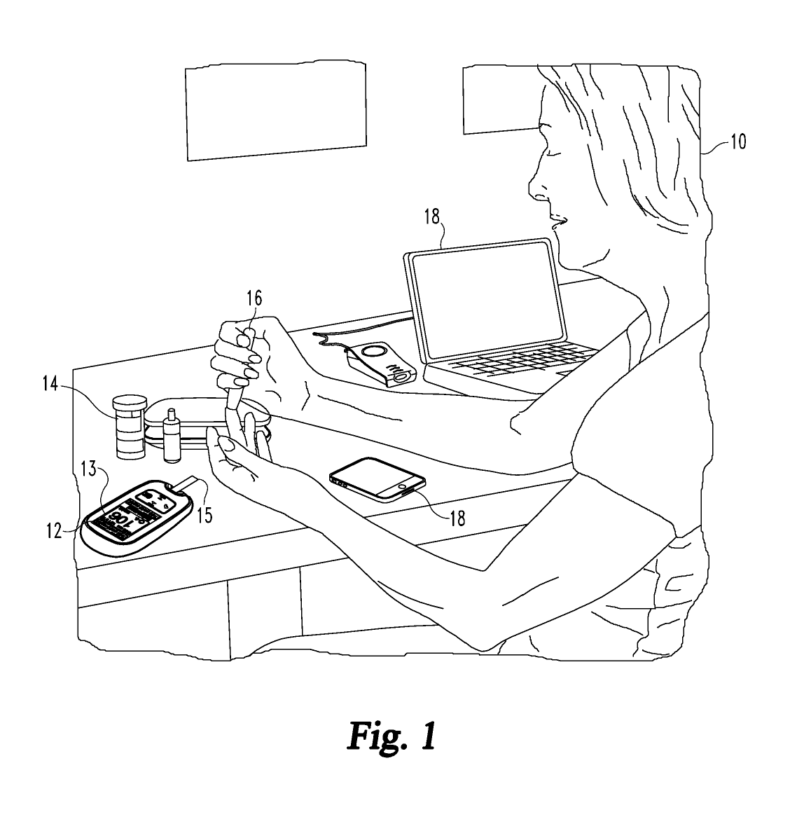

FIG. 1 shows a person with diabetes 10 in a self-testing environment with a handheld blood glucose meter 12, showing a displayed blood glucose measurement 13, test strip container 14, test strip 15, lancet 16, and computing devices 18. The person with diabetes 10 is typically the user 10 of the blood glucose meter 12; however, the user 10 can also be a clinician, health care provider, family member, or other person. The meter 12 is operated by inserting a disposable test strip 15 into the meter 12. The user lances typically a finger to obtain a small drop of blood that is placed on a test strip 15 collection site. The meter 12 performs electrochemical analysis of the blood and displays the blood glucose measurement 13. A current displayed blood glucose measurement 13 is used for therapy decisions such as insulin dosage and carbohydrate consumption. Typically, a person with diabetes 10 will have a blood glucose target range, and a blood glucose measurement outside of the target range such as higher than a target range or lower than a target range will be particularly relevant to making therapy adjustments. Past or retrospective blood glucose measurements are useful for providing insights regarding flagged or labeled events that can influence blood glucose measurements such as whether the blood glucose measurements were taken before meal or after meal. Other events that can influence blood glucose measurements may or may not be flagged such as emotional state, illness, exercise, and medication changes. If these events are not flagged, the person with diabetes 10 often has awareness of these events through their personal memory or through other records such as a personal calendar. When a person with diabetes 10 has an appointment with a clinician to assess therapy, past blood glucose measurements can easily be filtered in a variety of ways to aid in the understanding of both flagged and not flagged circumstances adversely affecting the person's therapy and potential changes to improve the therapy.

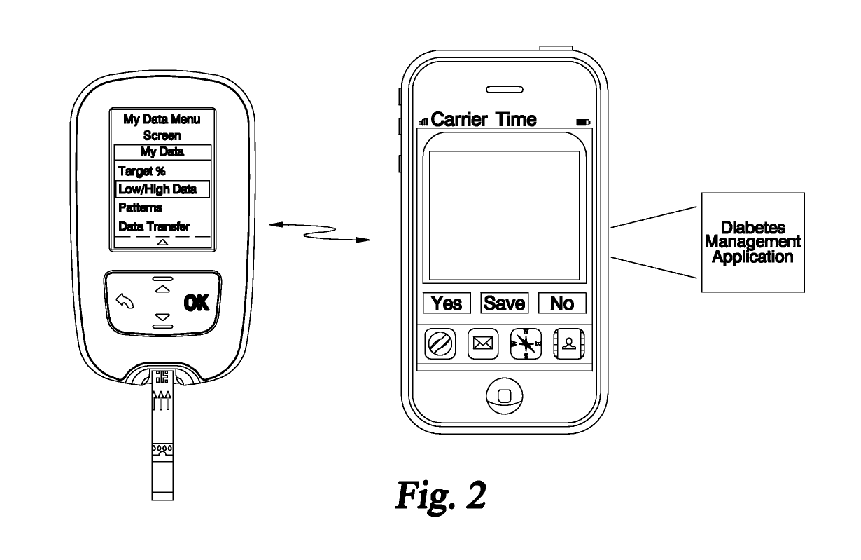

FIG. 2 shows a handheld blood glucose meter 12 and a computing device 18, shown as a mobile phone, with a diabetes management application 20. The blood glucose meter or bG meter 12 comprises a meter housing 22, meter display 24, meter user input buttons 26, a test strip port 28 and a test strip 15. The meter display 24 functions to display information for the user 10 to interpret and to highlight for selection and can be a dot matrix black and white display, a liquid crystal display (LCD), a Thin-Film-Transistor (TFT) LCD, and the like. The meter 12 user input buttons 26 include an up-scroll button 32, a down-scroll button 34, a selection button 36, and a back button 38. The up-scroll button 32 and down-scroll button 34 function to move to and highlight predetermined portions of the meter display 24 that can then be selected with the selection button 36. The up-scroll button 32 and down-scroll button 34 also function to scroll through blood glucose measurements presented on the meter display 24. The selection button 36, sometimes referred to as a confirmation button, generally functions for the user 10 to select a highlighted entry by instructing the meter 12 to store the highlighted entry in memory for further operations by the processor. The back button 38 functions to move to the previous screen, so the user 10 has the opportunity to change the highlighted information. The meter 12 is capable of wirelessly transmitting blood glucose records to a computing device 18 such as a mobile phone, a tablet computer, or a personal computer. An example of transferring information from a meter 12 to a computing device 18 is shown in U.S. patent application Ser. No. 13/794,985, Transferring Blood Glucose Measures Seamlessly from a Handheld Glucose Meter, filed Mar. 12, 2013, assigned to Roche Diagnostics Operations, Inc.

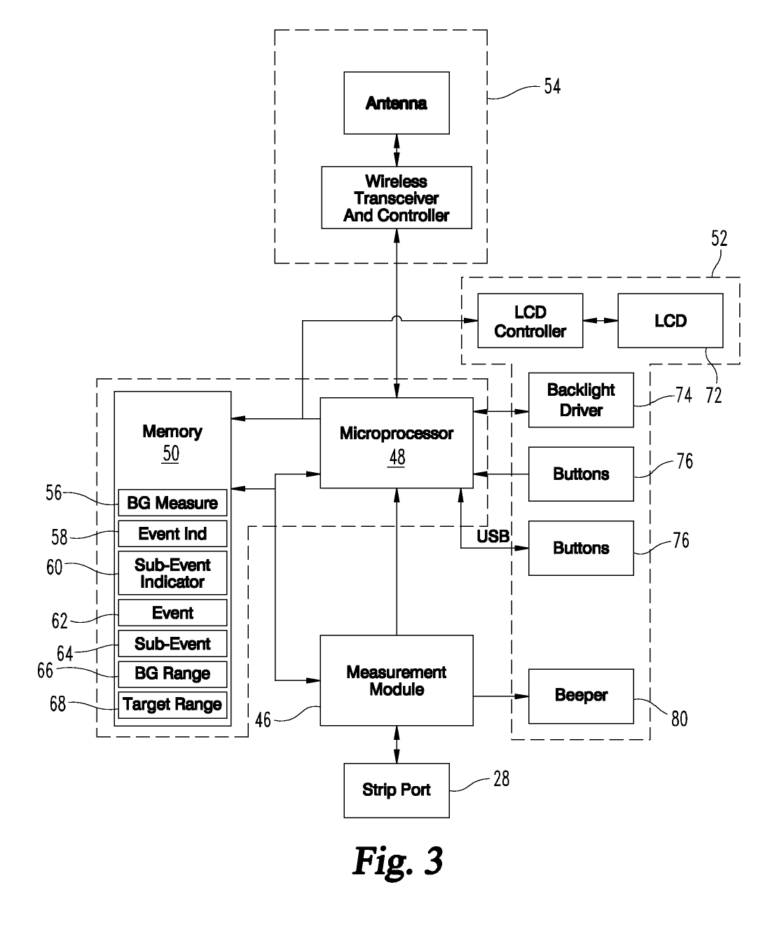

The computing device 18 generally has components that correspond to the handheld blood glucose meter 12 however without a strip port 28 and the capability to perform blood glucose measurements 56 (FIG. 3). The computing device 18 can be a mobile phone, a tablet computer, a laptop computer, or a desktop computer. The computing device 18 comprises a device housing 40, a device display 42, device user input buttons 44, and a diabetes management application 20. The computing device 18 is capable of wirelessly or via USB connection receiving blood glucose results from the meter 12. An example of blood glucose meter 12 or mobile phone 18 that performs blood glucose level pattern recognition is shown in U.S. patent application Ser. No. 13/936,535, Reminder, Classification, and Pattern Identification Systems and Methods for Handheld Diabetes Management Devices, filed Jul. 8, 2013, assigned to Roche Diagnostics Operations, Inc., which is hereby incorporated by reference. An example of a blood glucose meter 12 or mobile phone 18 that assesses a user's risk for hypoglycemia and hyperglycemia is shown in U.S. patent application Ser. No. 14/039,762, High/Low Blood Glucose Risk Assessment Systems and Methods, filed Sep. 27, 2013, assigned to Roche Diagnostics Operations, Inc., which is hereby incorporated by reference. An example of a diabetes management application 20 for a computing device is shown in U.S. Patent Pub. No. 2013/0172688, Diabetes Management Application for Mobile Phone, published Jul. 4, 2013, assigned to Roche Diagnostics Operations, Inc.

FIG. 3 shows a block diagram of a handheld blood glucose meter 12. The meter 12 comprises a strip port 28, a measurement module 46, a meter microprocessor 48, meter memory 50, meter user interface features 52, and a meter wireless transceiver 54. The meter memory 50 comprises a blood glucose (bG) measurement 56, event indicator 58, sub-event indicator 60, event 62, sub-event 64, bG range 66, and target range 68. The meter user interface features 52 comprise a meter display 72, such as liquid crystal display (LCD) controller and LCD, a meter backlight driver 74, meter buttons 76, and a beeper 80. The wireless transceiver 54 can be a Bluetooth low energy radio coupled to an antenna that communicates using a Continua Alliance compliant protocol such as described in U.S. patent application Ser. No. 14/155,954, Low Energy wireless Communication Systems and Methods for Medical Devices, filed Jan. 15, 2014, assigned to Roche Diagnostics Operations, Inc.

Blood glucose measurements 56 are stored in memory 50 such as a circular buffer that can hold blood glucose records indefinitely provided there is storage space before beginning to write over the oldest blood glucose measurements 56 that exceed the storage limit. For example, storage space hold a maximum of 500 blood glucose measurements 56 before beginning to write over the oldest blood glucose measurements 56. Blood glucose measurements 56 include a sequence number, date and time, and can also include additional contextual information such as related to a target range 68, events 62 and sub-events 64. If the target range function is enabled, the blood glucose measurements 56 can also include a bit fill or flag representing whether the blood glucose measurement 56 was within the target range, higher than the target range, lower than the target range, hyperglycemic, or hypoglycemic. The blood glucose measurement 56 contextual information for an event 62 or sub-event 64 can also be represented with a bit fill or flag.

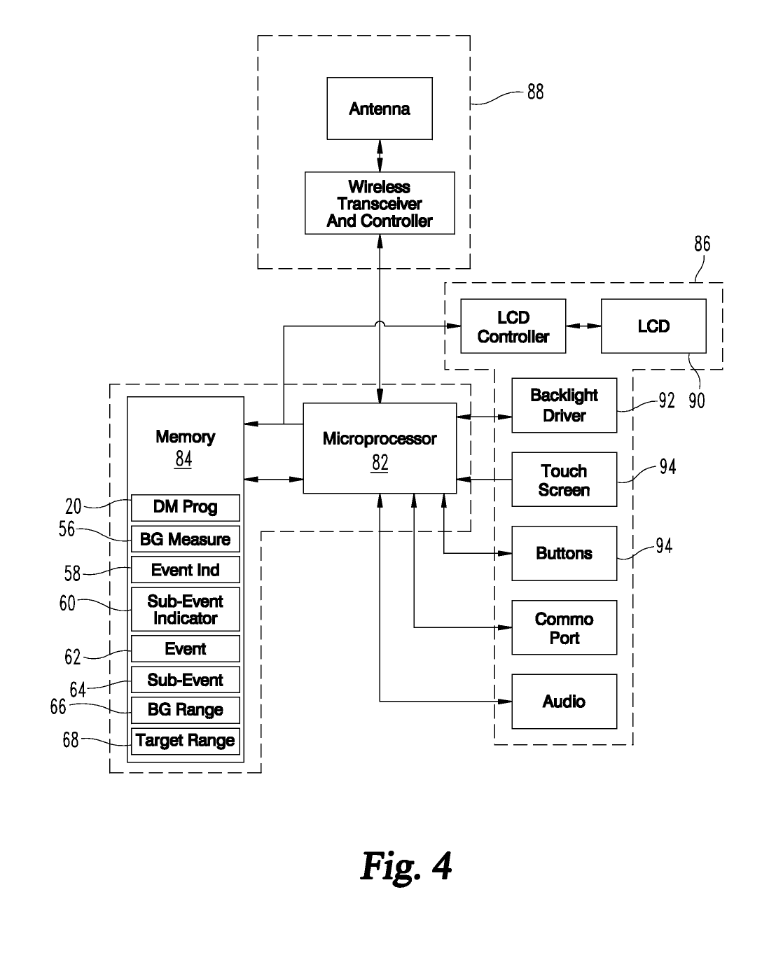

FIG. 4 shows a block diagram of a computing device 18. The computing device 18 comprises a device microprocessor 82, device memory 84, device user interface features 86, a device wireless transceiver 88, and a diabetes management program 20. The device memory 84 comprises a blood glucose measurement 56, event indicator 58, sub-event indicator 60, event 62, sub-event 64, bG range 66, and target range 68. The device 18 user interface features 86 comprise a device display 90, device backlight driver 92, device buttons 94. The wireless transceiver 88 can be a Bluetooth low energy radio coupled to an antenna that, after pairing, communicates with the blood glucose meter using a Continua Alliance compliant protocol.

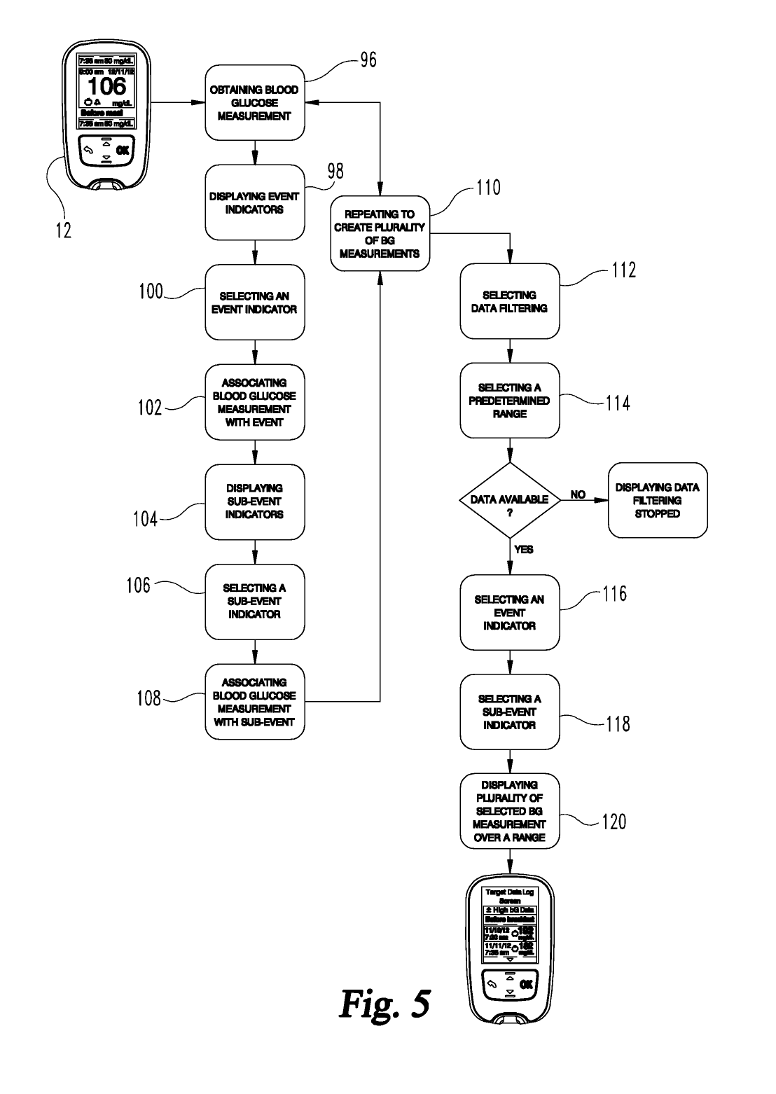

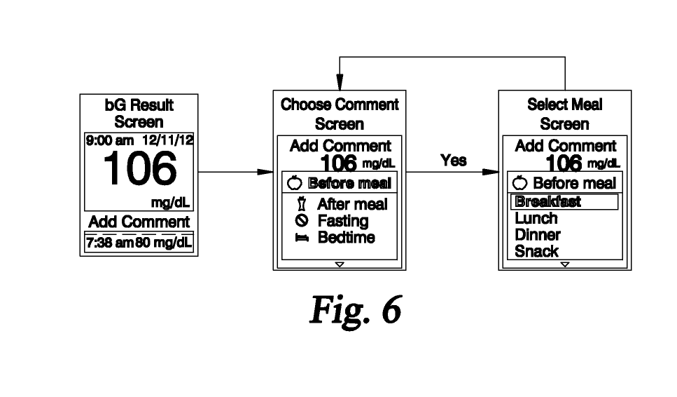

FIG. 5 shows a flowchart of a method for filtering blood glucose measurements 56 and associated contextual data of a user 10 on a handheld blood glucose meter 12 embodiment. The method comprises obtaining a blood glucose measurement 96, displaying a plurality of event indicators 98, selecting an event indicator 100, associating the blood glucose measurement 56 with an event 102, displaying a plurality of sub-event indicators 104, selecting a sub-event indicator 106, associating the blood glucose measurement 56 with a sub-event 108, repeating the previous elements to create a plurality of blood glucose measurements 110 with associated events 62 and sub-events 64, selecting data filtering 112, selecting a predetermined range of blood glucose measurements 114, selecting one of the event indicators 116, selecting one of the sub-event indicators 118, and displaying a plurality of blood glucose measurements 56 within the predetermined range 120. A simplified portion of the above process is shown in FIG. 6 with certain related user interface 26 navigation screens for selecting an event 62 and a sub-event 64.

Creating Blood Glucose Measurement with Contextual Data

The user 10 obtains a blood glucose measurement 56 with a blood glucose meter 12 having a meter display 24. The user 10 inserts a test strip 15 in the strip port 28 of the blood glucose meter 12, lances typically a finger, and places a small drop of blood on the test strip 30. The test strip 30 reacts chemically with the blood, and the measurement module 46 performs an electro-chemical or photometric analysis of the strip 15 to determine the blood glucose measurement 56. The blood glucose measurement 56 is generated by the measurement module 46 and the date and time along with the sequence number are added.

After performing the blood glucose measurement 56, the meter processor 48 reads the blood glucose measurement from the measurement module 46, writes the blood glucose measurement 56 to memory 50 and writes a glucose measurement for display 13 to the meter display 24 that includes a comments field. The user operates the up-scroll button 32 or down-scroll button 34 to highlight the comment field. The user then operates the selection button 36 to request the meter processor 48 to read from memory 50 a plurality of event indicators 58 that are then written to the meter display 24. Each of the event indicators 58 is associated with set of sub-event indicators 60 in memory 50 that are not yet displayed. The event indicator 58 appears on the meter display and represents events 62 that are relevant for better understanding of conditions when blood glucose was measured. Examples of event indicators 58 are before meal, after meal, fasting, bedtime, felt hypoglycemic, exercise, alcohol, and the like. The user 10 operates the up-scroll button 32 or down-scroll button 34 to highlight the desired event indicator 58. The user 10 then operates the selection button 36 to request the meter processor 48 write the event 62 to memory 50 that is associated with the event indicator 58. The meter processor 48 associates the blood glucose measurement 56 with an event 62 that corresponds to the event indicator 58, and stores the event 62 and the event's association with the blood glucose measurement 56 in the meter's memory 50.

The meter processor 48 then reads from memory 50 and writes to the meter display 24 a plurality of sub-event indicators 60 associated with the selected event indicator 60 that provide further relevant information for a better understanding of the conditions or context of the blood glucose measurement 56. The user 10 operates the up-scroll button 32 or down-scroll button 34 to highlight the desired sub-event indicator 60. The previously selected event indicators 58 of before meal and after meal each have the associated sub-event indicator 60 selected from one of breakfast, lunch, dinner, and snack. The selected event indicator 58 of exercise has the associated sub-event indicators 60 selected from one of walking, running, cycling, and swimming. The user 10 then operates the selection button 36 to request the meter processor 48 to write the sub-event 64 to memory 50 that is associated with the sub-event indicator 60. The processor 48 associates the blood glucose measurement 56 with a sub-event 64 that corresponds to the sub-event indicator 60, and stores the sub-event 64 association with the blood glucose measurement 56 in the meter's memory 50. Table 1 below summarizes the previously described events 62 and associated with sub-events 64 provides additional events 62 and sub-events 64 along with their association.

TABLE 1

| Events | Sub-Events |
|---|---|
| Before Meal | Breakfast |
| | Lunch |
| | Dinner |
| | Snack |
| | Coffee Break |
| After Meal | Breakfast |
| | Lunch |
| | Dinner |
| | Snack |
| | Coffee Break |
| Exercise | Walking |
| | Running |
| | Cycling |
| | Swimming |

The user 10 repeats the steps of obtaining a blood glucose measurement 96, displaying a plurality of event indicators 98, selecting an event indicator 100, associating the blood glucose measurement 56 with an event 102, displaying a plurality of sub-event indicators 104, selecting a sub-event indicator 106, and associating the blood glucose measurement 56 with a sub-event 108. By repeating these steps 110, the user creates a plurality of blood glucose measurements 110 with associated events 62 and 64 sub-events.

Filtering Blood Glucose Measurements with Contextual Data

The user 10 begins the process of filtering blood glucose measurements 56 from the meter 12 main menu by highlighting and selecting My Data that displays data filtering 112 with a display indicator such as “Low/High Data.” The meter 12 then displays predetermined ranges 116 of blood glucose measurements 56 such as high data, low data, hypoglycemic and hyperglycemic. In one embodiment, the predetermined range 116 is selected from one of a low data range that is below a target range and a high data range that is above a target range. The low data range selected from within a first range from about 50 mg/dL to about 100 mg/dL, and the high data range selected from within a second range from about 101 mg/dl to about 200 mg/dl. Table 2 below summarizes the previously described predetermined range for blood glucose measurements 56 and provides additional predetermined ranges.

TABLE 2

| Predetermined Range | Blood Glucose Range |
|---|---|
| Low Data | Below Target |
| High Data | Above Target |
| Hypoglycemic | Below Target and |
| | Hypoglycemic Limit |
| Hyperglycemic | Above Target and |
| | Hyperglycemic Limit |

The user 10 operates the up-scroll button 32 or down-scroll button 34 to highlight the desired predetermined range of blood glucose measurements 56. The user then operates the selection button 36 to request the meter processor 48 to write the highlighted predetermined range to memory 50 for operation by the processor 48.

The meter processor 48 then writes to the display 24 event indicators 58 available for selection. The user 10 operates the up-scroll button 32 or down-scroll button 34 to highlight the desired event indicator 58. The user then operates the selection button 36 to request the meter processor 48 to write the event 62 associated with the event indicator 58 to memory 50 for operation by the processor 48. The meter processor 48 then writes to the display 24 sub-event indicators 60 available for selection. The user 10 operates the up-scroll button 32 or down-scroll button 34 to highlight the desired sub-event indicator 60. The user 10 then operates the selection button 36 to request the meter processor 48 to write the sub-event 64 associated with the sub-event indicator 60 to memory 50 for operation by the processor 48.

The meter 12 displays a plurality of blood glucose measurements 118 within the predetermined range that have both the selected event 62 and the selected sub-event 64 in a tabular format. This plurality of blood glucose measurements 118 with contextual data is not typically stored in memory to ensure that the measurements 118 are current. In addition to the event 62, sub-event 64, and predetermined range 116, the meter 12 can be configured to show blood glucose measurements over a time period such as 90 days. The plurality of blood glucose measurements 56 displayed would typically be ordered with the most recent blood glucose measurement 56 meeting the selection criteria displayed first, and the user would scroll through the older blood glucose measurements 56.

In another embodiment, the blood glucose meter 12 does not need to create a plurality of blood glucose measurements 110 with associated events 62 and sub-events 64 because they are already stored in meter memory 50. In this embodiment, the user 10 would begin the process of filtering blood glucose measurements 56 from the meter main menu by highlighting and selecting My Data and follow the process discussed above.

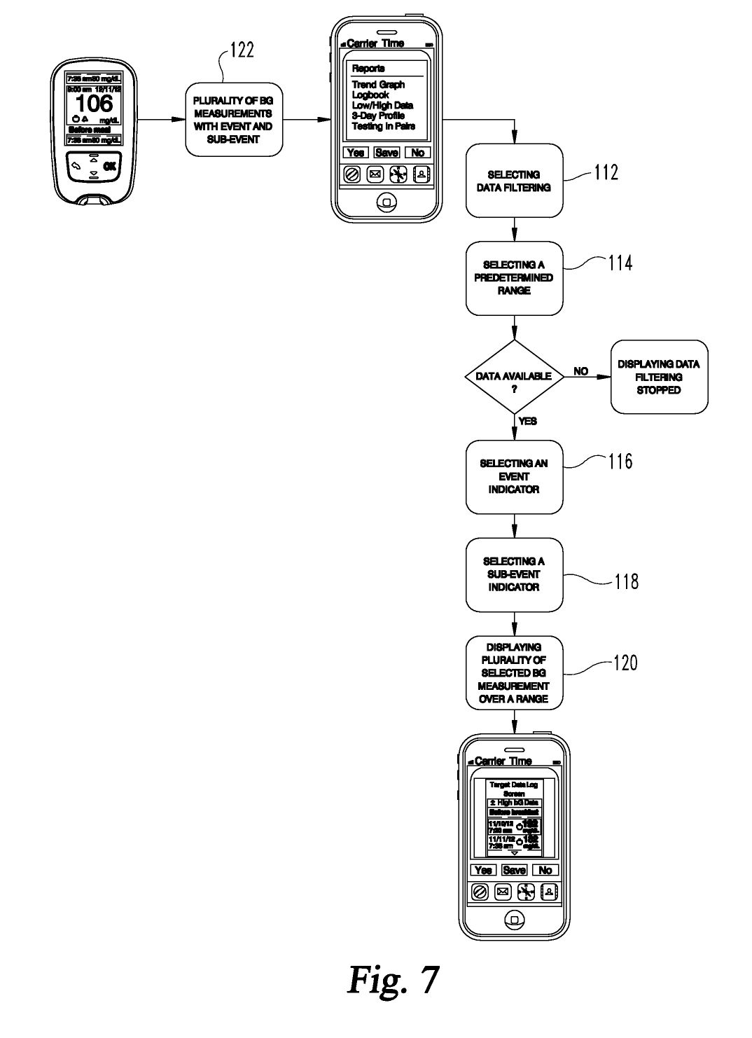

FIG. 7 shows a flowchart of a method for filtering blood glucose measurements 56 and associated contextual data of a user 10 on a computing device 18 embodiment. In another embodiment, the blood glucose meter 12 does not need to create a plurality of blood glucose measurements 110 with associated events 62 and sub-events 64 because they are already stored in meter memory 50. The meter 12 transmits these blood glucose measurements 122 to a computing device 18, such as a mobile phone, tablet or other computer, and the computing device 18 has a diabetes management application 20 that performs the remaining elements. The user 10 would operate the computing device 18 according to the computing device's user interface 86 to select data filtering 112, select a predetermined range of blood glucose measurement 116, select an event indicator 100, select as associated sub-event indicator 106 to display a blood glucose measurements 13 within the selected predetermined range 116 that have both the associated event 62 and sub-event 64.

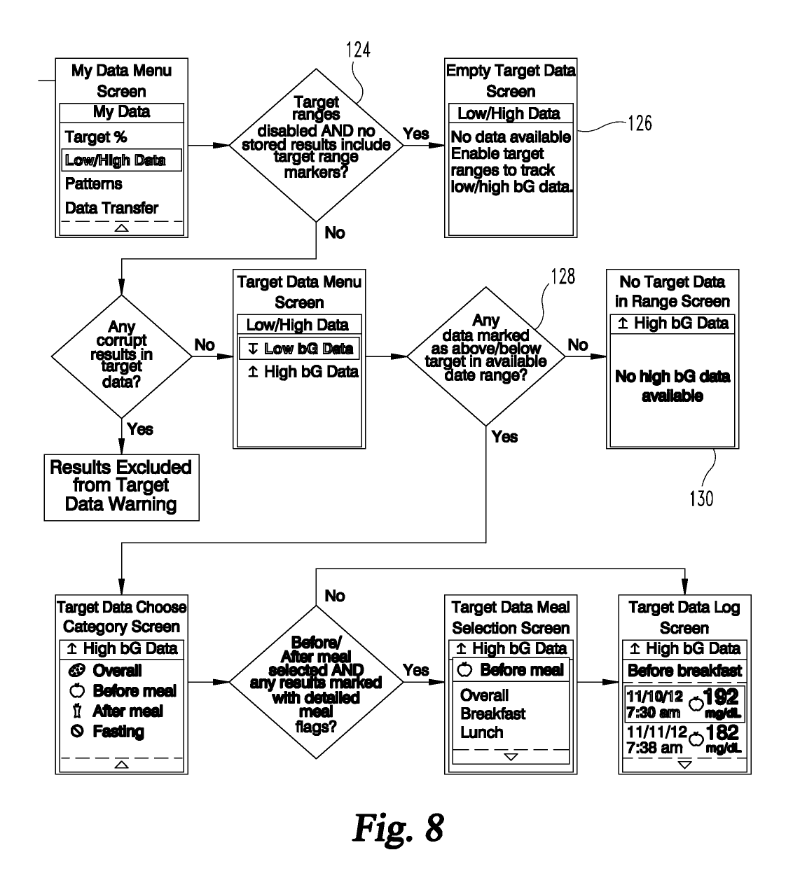

FIG. 8 shows certain user interface navigation screens related to stopping the contextual data filtering process. Under certain conditions such as when the blood glucose measurement 56 target range 68 is disabled or if there are no stored blood glucose measurements 56 within the predetermined range, the blood glucose meter 12 or computing device 18 will not display a plurality of blood glucose measurements 56 in the predetermined range. To reduce user irritation under these conditions, the blood glucose meter 12 or computing device 18 will halt data filtering and return to a home screen. If the blood glucose meter 12 or computing device 18 continued with data filtering, the user 10 would waste time by performing more actions when there will be no blood glucose measurements 56 that meet the selection criteria. If the user selects the predetermined range on the meter display and the meter determines that the target range is disabled 124 and there are no stored blood glucose measurements 56 having target range markers, data filtering is stopped 126 because blood glucose measurements 56 will meet the selection criteria. If the user selects the predetermined range and there are no stored blood glucose measurements 56 within the predetermined range 128, data filtering is stopped 130 because no blood glucose measurements 56 will meet the selection criteria.

Thus, embodiments of the handheld blood glucose meter with contextual data filtering are disclosed. One skilled in the art will appreciate that the teachings can be practiced with embodiments other than those disclosed. The disclosed embodiments are presented for purposes of illustration and not limitation, and the invention is only limited by the claims that follow.

What is claimed is:

1. A method for filtering blood glucose measurements and associated contextual data of a user on a handheld blood glucose meter, comprising:
storing by the blood glucose meter a plurality of blood glucose measurements with each of the blood glucose measurements having an associated date and time, an event, and a sub-event associated with the event, the blood glucose having a meter display;
selecting data filtering on the meter display by the user;
selecting a predetermined range for blood glucose measurements;
selecting from a plurality of event indicators on the meter display an event indicator by the user;
selecting from a plurality of sub-event indicators on the meter display associated with the selected event indicator a sub-event indicator by the user; and
displaying a plurality of blood glucose measurements within the predetermined range that have both the event and sub-event that correspond to the event indicator and sub-event indicator selected by the user.

2. The method of claim 1, wherein the predetermined range is selected from one of a low data range that is below a target range and a high data range that is above the target range.

3. The method of claim 2, wherein the low data range is selected from within a first range from about 50 mg/dL to about 100 mg/dL and the high data is selected from within a second range from about 101 mg/dL to about 200 mg/dL.

4. The method of claim 1, wherein the event indicator is selected from one of before meal, after meal, fasting, bedtime, felt hypoglycemic, exercise, and alcohol.

5. The method of claim 1, wherein the selected event indicators of before meal, after meal, and exercise each have associated sub-event indicators selected from one of breakfast, lunch, dinner, snack, coffee break, walking, running, cycling, and swimming.

6. The method of claim 1, further comprising:
determining after the user selects the predetermined range on the meter display that a target range is disabled and no stored blood glucose measurements have target range markers;
stopping data filtering because no stored blood glucose measurements have target range markers;
displaying that data filtering has been stopped because no stored blood glucose measurements have target range markers on the meter display.

7. A method for filtering blood glucose measurements and associated contextual data of a user on a computing device, comprising:
receiving by the computing device from a blood glucose meter a plurality of blood glucose measurements, each of the blood glucose measurements having an associated date and time, an event, and a sub-event associated with the event, the computing device having a device display;
selecting data filtering on the computing device display by the user;
selecting a predetermined range for blood glucose measurements;
selecting from a plurality of event indicators on the computing device display an event indicator by the user;
selecting from a plurality of sub-event indicators on the computing device display associated with the selected event indicator a sub-event indicator by the user; and
displaying a plurality of blood glucose measurements within the predetermined range that have both the event and the sub-event that correspond to the event indicator and sub-event indicator selected by the user.

8. The method of claim 7, wherein the predetermined range is selected from one of a low data range that is below a target range and a high data range that is above the target range.

9. The method of claim 8, wherein the low data range is selected from within a first data range from about 50 mg/dL to about 100 mg/dL and the high data range is selected from within a second data range from about 101 mg/dL to about 200 mg/dL.

10. The method of claim 7, wherein the event indicator is selected from one of before meal, after meal, fasting, bedtime, felt hypoglycemic, exercise, and alcohol.

11. The method of claim 7, wherein the selected event indicators of before meal, after meal, and exercise each have associated sub-event indicators selected from one of breakfast, lunch, dinner, snack, coffee break, walking, running, cycling, and swimming.

12. The method of claim 7, further comprising:
determining after the user selects the predetermined range of blood glucose measurements on the computing device display that a target range is disabled and no stored blood glucose measurements have target range markers;
stopping data filtering because no blood glucose measurements have target range markers;
displaying that data filtering has been stopped because no blood glucose measurements have target range markers on the computing device display.

13. A method for filtering blood glucose measurements and associated contextual data of a user on a handheld blood glucose meter, comprising:
(a) obtaining a blood glucose measurement with a blood glucose meter operated by the user and the blood glucose meter associates a date and time with the blood glucose measurement, the blood glucose meter having a display;
(b) displaying a plurality of event indicators on the meter display;
(c) selecting an event indicator by the user from the plurality of event indicators;
(d) associating the blood glucose measurement with an event that corresponds to the event indicator;
(e) displaying a plurality of sub-event indicators on the meter display associated with the selected event indicator;
(f) selecting a sub-event indicator by the user from the plurality of sub-event indicators;
(g) associating the blood glucose measurement with a sub-event corresponding to the selected sub-event indicator;
repeating (a)-(g) by the user to create a plurality of blood glucose measurements with associated events and sub-events;
selecting data filtering on the meter display by the user;
selecting a predetermined range for blood glucose measurements;
selecting from the plurality of event indicators on the meter display an event indicator by the user;
selecting from the plurality of sub-event indicators on the meter display associated with the selected event indicator by the user a sub-event indicator by the user; and
displaying a plurality of blood glucose measurements within the predetermined range that have both the event and the sub-event that correspond to the selected event indicator and the selected sub-event indicator selected by the user.

14. The method of claim 13, wherein the predetermined range is selected from one of a low data range that is below a target range and a high data range that is above a target range.

15. The method of claim 14, wherein the low data range is selected from within a first range from about 50 mg/dL to about 100 mg/dL and the high data range is selected from within a second range from about 101 mg/dL to about 200 mg/dL.

16. The method of claim 13, wherein the event indicator is selected from one of before meal, after meal, fasting, bedtime, felt hypoglycemic, exercise, and alcohol.

17. The method of claim 13, wherein the selected event indicators of before meal, after meal, and exercise each have associated sub-event indicators selected from one of breakfast, lunch, dinner, snack, coffee break, walking, running, cycling, and swimming.

18. The method of claim 13, further comprising:
determining after the user selects the predetermined range on the meter display that a target range is disabled and no stored blood glucose measurements have target range markers;
stopping data filtering because no stored blood glucose measurement have target range markers; and
displaying that data filtering has been stopped because no stored blood glucose measurements have target range markers on the meter display.
